(19) United States
(12) Patent Application Publication
(10) Pub. No.: US 2004/0138634 A1
(43) Pub. Date: Jul. 15, 2004

(54) Absorbent article having improved softness

(76) Inventors: John D. Litvay, Downingtown, PA (US); Ebba A. Hansen, Cumming, GA (US); Pamela Peters, Harleysville, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/339,662

(22) Filed: Jan. 10, 2003

(51) Int. Cl. A61F 13/15; A61F 13/20

(52) U.S. Cl. 604/374

(57) ABSTRACT

The present invention generally relates to absorbent articles and to methods for forming the absorbent articles. The absorbent articles of the present invention include an absorbent core that is disposed between a top sheet and a backsheet, whereby the articles have improved stiffness and an improved stiffness index.

### FIELD OF THE INVENTION

The present invention relates generally to absorbent articles having improved softness, and specifically to absorbent articles that retain their softness even after being subjected to compressive stresses generated during packaging and storage of a plurality of articles. The absorbent articles of the present invention include an absorbent core that is disposed between at least partially between a top sheet and a back sheet, whereby the articles have improved stiffness and an improved stiffness index.

### BACKGROUND OF THE INVENTION

Disposable absorbent garments such as infant diapers or training pants, adult incontinence products and other such products, typically are constructed with a moisture-impervious outer backing sheet, a moisture-previous body-contacting inner liner sheet, and a moisture-absorbent core disposed between the liner and backing sheets. Significant efforts have been undertaken to find cost-effective materials for use in absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining even under moderate pressure large quantities of liquid, such as water and body wastes, relative to their own weight.

One of the more important features of a disposable absorbent garment is softness. Softness can be measured objectively by a number of physical characteristics, but consumer perceived softness may be the most important characteristic of them all. Most disposable absorbent articles are marketed in packs containing a plurality of tightly packed articles. These absorbent articles are subjected to compressive stresses during packaging and shipping that can have a negative impact on the perceived softness of the absorbent article. For example, conventional fluff core diapers are somewhat rigid when removed from the packaging due to their lack of ability to rebound from the compressive stresses generated during packaging and shipping.

The conventional fluff core diapers also may suffer from select hardened areas that may have been caused by compression during the manufacturing process. The compressed pulp core also my have stiff ridges along the fold lines in the ears and crotch area. The fluff cores oftentimes maintain some degree of rigidity when worn by the user.

Softness of tissues typically is measured by the stiffness of the tissue. For example, U.S. Pat. Nos. 5,772,845 and 5607,551, the disclosures of which are incorporated by reference herein in their entirety, disclose soft tissues having low stiffness. Low stiffness typically translates into greater flexibility and accordingly, improved softness.

It also is known to measure the flexure resistance as a measure of stiffness as an indicator of softness. U.S. Pat. No. 5,009,653, (“the '653 patent”) the disclosure of which is incorporated by reference herein in its entirety, discloses a method of measuring the flexure resistance of a sanitary napkin. The '653 patent discloses a sanitary napkin having a total capacity of at least about 14.0 grams, and a flexural resistance of less than about 300 grams, when measured using the testing protocol described therein.

It is known to provide absorbent laminates comprised of, for example, an upper layer, a lower layer, and a central fibrous layer containing from 50% to 95% by weight SAP. U.S. Pat. No. 6,068,620, the disclosure of which is incorporated herein by reference in its entirety, discloses that the upper and lower layers are comprised of tissue, airlaid fluff pulp or synthetic non-woven fibrous layers. The upper and lower layers are said to assist in maintaining the integrity of the core, the laminate layered arrangement is said to minimize gel blocking, and the laminate can be folded in various configurations. It also is known to provide absorbent cores comprised of differing materials in an attempt to maximize comfort and efficiency of the core, and to provide areas having varying degrees of absorbency. U.S. Pat. No. 5,849,002, the disclosure of which is incorporated by reference herein in its entirety, discloses absorbent cores having three zones: (i) one zone for receiving fluids; (ii) one zone for distributing and storing fluids; and (iii) one zone for preventing leakage.

The description herein of advantages and disadvantages of various features, embodiments, methods, and apparatus disclosed in other publications is in no way intended to limit the present invention. Indeed, certain features of the invention may be capable of overcoming certain disadvantages, while still retaining some or all of the features, embodiments, methods, and apparatus disclosed therein.

### SUMMARY OF THE INVENTION

It would be desirable to provide an absorbent garment having an improved softness even after the garment has been stored and transported. It also would be desirable to provide an absorbent garment having good leakage prevention properties, while at the same time provide for a soft article. A feature of the invention therefore is to provide an absorbent article having improved softness, when compared to conventional absorbent articles. It also is a feature of the invention to provide a pack of absorbent articles that retain their softness even after being folded, compressed, stored, and transported.

In accordance with these and other features of various embodiments of the present invention, there is provided an absorbent article having a top sheet, a back sheet, and an absorbent core at least partially disposed between the top sheet and the back sheet, whereby the absorbent article has a stiffness of less than about 900 gf.

In accordance with another feature of the invention, there is provided a plurality of absorbent articles that have been folded and arranged in a consumer pack, each absorbent article having a top sheet, a back sheet, and an absorbent core at least partially disposed between the top sheet and the back sheet, whereby the absorbent article has a stiffness of less than about 900 gf.

Another embodiments of the present invention includes an absorbent article having a top sheet, a back sheet, and an absorbent core at least partially disposed between the top sheet and the back sheet, whereby the absorbent article has a stiffness index (ratio of stiffness to total capacity )of less than about 1.5 gf/g.

In accordance with another feature of the invention, there is provided a plurality of absorbent articles that have been folded and arranged in a consumer pack, each absorbent article having a top sheet, a back sheet, and an absorbent core at least partially disposed between the top sheet and the back sheet, whereby the absorbent article has a has a stiffness index (ratio of stiffness to total capacity )of less than about 1.5 gf/g.

In preferred embodiments of the invention, the absorbent article is a diaper, and the stiffness is less than about 2,500 gf, and/or the stiffness index is less than about 4.5. In another preferred embodiment of the invention, the absorbent article is a training pant, or “pull up” diaper, and the stiffness is less than about 900 gf, and/or the stiffness index is less than about 1.5. In yet another preferred embodiment of the invention, the absorbent article is a swimming pant, or “swimmers” diaper, and the stiffness is less than about 800 gf, and/or the stiffness index is less than about 8.8.

In accordance with an additional feature of an embodiment of the invention, there is provided a method of making an absorbent article that includes providing to a garment forming station a top sheet and a back sheet. The method also include preparing an absorbent core by providing to a core forming station an upper layer, a lower layer, and central absorbent layer comprising a mixture of tow fibers and SAP. The central absorbent layer is formed at the core forming station whereby the tow fibers and SAP are mixed and disposed between the upper and lower layer to form the absorbent core. The method concludes by disposing the absorbent core between the top sheet and the back sheet. In accordance with the method, the absorbent article has a stiffness of less than about 900 gf, and/or a stiffness index of less than about 1.5.

In accordance with yet another feature of an embodiment of the invention, there is provided a method of making a pack of absorbent articles comprising making a plurality of absorbent articles in accordance with the method described immediately above. The plurality of absorbent articles then are folded, and assembled adjacent one another to form one or more rows, each containing a plurality of folded absorbent articles. The one or more rows containing a plurality of absorbent articles then are compressed and packaged. The absorbent articles have a stiffness of less than about 900 gf, and/or a stiffness index of less than about 1.5.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with elastic members fully stretched in the main portion of the garment;

FIG. 2 is a schematic of an apparatus useful in making an absorbent article in accordance with the present invention; and

FIG. 3 is an illustration of an apparatus useful in measuring the stiffness of an absorbent article in accordance with the present invention.

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. Preferably, the invention relates to diapers, training pants and swimmers. Swimmers in the context of the invention denotes articles intended for use while swimming or in contact with water. Consequently, swimmer products typically contain little or no superabsorbent polymer and have much lower total capacities, when compared to conventional diapers and training pants.

The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer” and “lower layer” that refer to the layers surrounding the absorbent core of the invention are used merely to describe one layer above the core, and one layer below the core. The upper layer need not always remain vertically above the core, and the lower layer need not always remain vertically below the core. Indeed, many embodiments of the invention encompass various configurations of the absorbent core whereby the core is folded in such a manner that the upper layer ultimately becomes the vertically highest and vertically lowest layer at the same time. Other configurations are contemplated within the context of the present invention.

The term “component” can refer, but is not limited to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved.

The groups of filaments from a plurality of spinnerets typically are combined into a tow which is then subjected to a drawing operation to impart the desired physical properties to the filaments comprising the tow. It is believed that tow adds surface area to the core, which improves capacity and capillarity as well as surfaces for glue to attach SAP. Tow also is believed to add wet integrity to the core that would otherwise be very poor, as well as add dry integrity that helps with the manufacturing processes.

The present invention relates generally to absorbent articles, and in particular to absorbent articles containing a top sheet, a back sheet, and an absorbent core at least partially disposed between the top sheet and the back sheet. The absorbent articles of the invention have a stiffness of less than about 900 gf, when measured in accordance with the flexural resistance test method described herein. Preferably, the absorbent articles also have a stiffness index, which is the ratio of the stiffness to the total capacity, of less than about 1.5 gf/g. The invention also relates to a method of making the above-described absorbent article that contains a top sheet, a back sheet, and an absorbent core disposed between the top sheet and the back sheet. The absorbent core of the invention preferably is comprised of at least three layers, whereby two of the layers are outer layers, (i.e., an upper and a lower layer) and the inner layers is a central fibrous layer containing tow fibers and SAP, or just tow fibers and substantially no SAP (i.e., less than about 10 wt % SAP) in the case of swimmer absorbent articles.

The present invention also encompasses a plurality of absorbent articles that have been folded and arranged in a consumer pack, each absorbent article having a top sheet, a back sheet, and an absorbent core at least partially disposed between the top sheet and the back sheet, whereby the absorbent article has a stiffness of less than about 900 gf. Preferably, the absorbent articles in the pack each have a stiffness index of less than about 1.5.

In preferred embodiments of the present invention, the absorbent article is a diaper having a stiffness of less than about 2,500 gf, and/or the stiffness index is less than about 4.5. In another preferred embodiment of the invention, the absorbent article is a training pant, or “pull up” diaper, and the stiffness is less than about 900 gf, and/or the stiffness index is less than about 1.5. In yet another preferred embodiment of the invention, the absorbent article is a swimming pant, or “swimmers” diaper, and the stiffness is less than about 800 gf, and/or the stiffness index is less than about 8.8.

The method of the invention includes providing to a garment forming station a top sheet and a back sheet, and preparing an absorbent core by providing to a core forming station an upper layer, a lower layer, and central absorbent layer comprising a mixture of tow fibers and SAP. The central absorbent layer is formed at the core forming station whereby the tow fibers and SAP are mixed and disposed between the upper and lower layer to form the absorbent core. The method concludes by disposing the absorbent core between the top sheet and the back sheet. In accordance with the method, the absorbent article has a stiffness of less than about 900 gf, and/or a stiffness index of less than about 1.5.

The invention also includes a method of making a pack of absorbent articles comprising making a plurality of absorbent articles in accordance with the method described immediately above. The plurality of absorbent articles then are folded, and assembled adjacent one another to form a single row of a plurality of folded absorbent articles. The single row of a plurality of absorbent articles then are compressed and packaged. The absorbent articles have a stiffness of less than about 900 gf, and/or a stiffness index of less than about 1.5.

The absorbent core of the present invention preferably comprises two outer layers, which may be made of the same single sheet or ply of material folded over onto itself, surrounding an inner absorbent layer. The inner absorbent layer preferably includes tow fibers and SAP, and may include up to 10 wt % conventional fluff pulp. The tow fibers preferably are cellulose acetate or polypropylene fibers, and the inner absorbent layer preferably contains more than 30% by weight SAP, based on the weight of the core.

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

Other non-SAP-containing roll good materials such as latex or thermally bonded airlaid fluff pulp, (e.g., roll good available from Walkisoft, Merfin or Fort James), or synthetic spunbonded, carded, or hydro-entangled non-woven may be positioned above and below the absorbent core. At least the central fibrous layer of the absorbent core preferably contains 30-95% by weight particulate or fibrous SAP and at least one other fibrous or particulate material that is comprised of tow fibers.

The invention now will be described with reference to the attached drawings illustrating preferred embodiments of the invention. For clarity, features that appear in more than one Figure have the same reference number in each Figure.

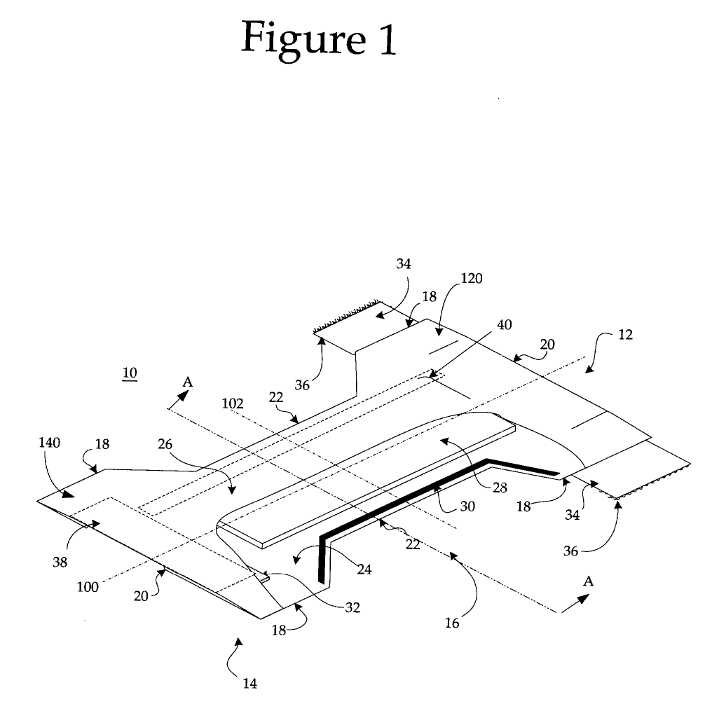

FIG. 1 is a partially cut away depiction of an exemplary embodiment of an absorbent garment 10 (preferably a disposable absorbent garment) that can be made in accordance with the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user. When folded to form a pack of absorbent articles, the article 10 is folded along the center of lateral axis 102, and also may be longitudinally folded, but typically not along the center axis 100. That is, if folded longitudinally, article 10 is folded such that the ear portions 120 and 140 are folded inward toward center axis 100.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer. If formed as a training pant, (or “pull-up” pant) first and second waist regions 12, 14 are already connected to one another, elastics typically are included in the ear portions 120, 140, and the user simply pulls-up the article until fitted around his/her waist.

The garment 10 preferably comprises a top sheet material 24, (“top sheet”) and a back sheet material 26, (“back sheet”) which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14.

Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. The invention is not intended to be limited to any specific materials for these components. The top sheet 24 and back sheet can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable materials include non-woven spunbond or carded webs of polypropylene, polyethylene, nylon, polyester and blends of these materials.

The back sheet 26 may be covered with or laminated with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The top sheet 24, and the back sheet 26 may be formed of three separate portions or panels. Those skilled in the art will recognize, however, that top sheet 24 need not be made of three separate panels, and that it may be comprised of one unitary item. A first top sheet panel may comprise a central top sheet panel formed from preferably a liquid-pervious material that is either hydrophobic or hydrophilic. The central top sheet panel preferably extends from substantially the second waist region 14 to the first waist region 12, or a portion thereof. The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

The standing leg gather(s) preferably are disposed such that they extend laterally away from the surface of top sheet 24. Standing leg gather(s) 40 may be treated with a suitable surfactant to modify their hydrophobicity/hydrophilicity as desired, and they may be treated with skin wellness ingredients to reduce skin irritation. Alternatively, the standing leg gather(s) 40 may be formed as separate elements and then attached to the body side liner. The standing leg gather(s) 40 preferably include a portion that folds over onto itself to form a small enclosure. At least one, and depending on the size of the enclosure sometimes more than one, elastic member may be secured in the enclosure in a stretched condition. It is known that when the flap elastic attempts to assume the relaxed, unstretched condition, the standing leg gather(s) 40 rise above the surface of the top sheet 24.

The top sheet 24 may be made of any suitable relatively liquid-pervious material currently known in the art or later discovered that permits passage of a liquid therethrough. Examples of suitable top sheet materials include nonwoven spun-bonded or carded webs of polypropylene, polyethylene, nylon, polyester and blends of these materials, perforated, apertured, or reticulated films, and the like. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make second and third top sheet panels, preferably are substantially fluid impervious and hydrophobic, while the remainder of the top sheet 24 (e.g., central top sheet panel) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the top sheet 24 to the back sheet 26. The particular joining method may be dictated by the types of materials selected for the top sheet 24 and back sheet 26. The particular methods of joining the respective components together can impact the stiffness of the article. For example, if too much adhesive is used to join top sheet 24 to back sheet 26, the excess adhesive when dried on the component may impart an additional stiffness. It therefore is preferred in the invention to control the amount of bonding material so as not to adversely affect the stiffness.

As mentioned above, absorbent garment preferably is provided with leg elastics 30 extending through crotch region 16, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches wide and about 3-6 inches long. The foam strips preferably are positioned between the top sheet 24 and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably are stretched 50-150%, preferably 100% more than their unstretched dimension before being adhesively secured between the back sheet 26 and top sheet 24.

Each edge 22 that forms the leg openings preferably is provided with an adjacent leg elastic containment system 30. In the preferred embodiment, three strands of elastic threads are positioned to extend adjacent to leg openings between the outer top sheet portions or panels and the back sheet 26. Any suitable elastomeric material can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (DuPont), GLOSPAN (Globe) or SYSTEM 7000 (Fulflex).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. Alternatively, other absorbent article fastening systems are also possible, including safety pins, buttons, and snaps.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products.

The underlying structure beneath the top sheet 24 may include, depending on the diaper construction, various combinations of elements, but in each embodiment, it is contemplated that the absorbent garment will preferably include an absorbent core 28 positioned between the top sheet 24 and back sheet 26. The absorbent core 28 may take a number of different constructions, depending on how it is configured. The absorbent core 28 preferably contains a central fibrous layer preferably containing about 50-95% by weight SAP, and at least tow fibers, surrounded by upper and lower layers.

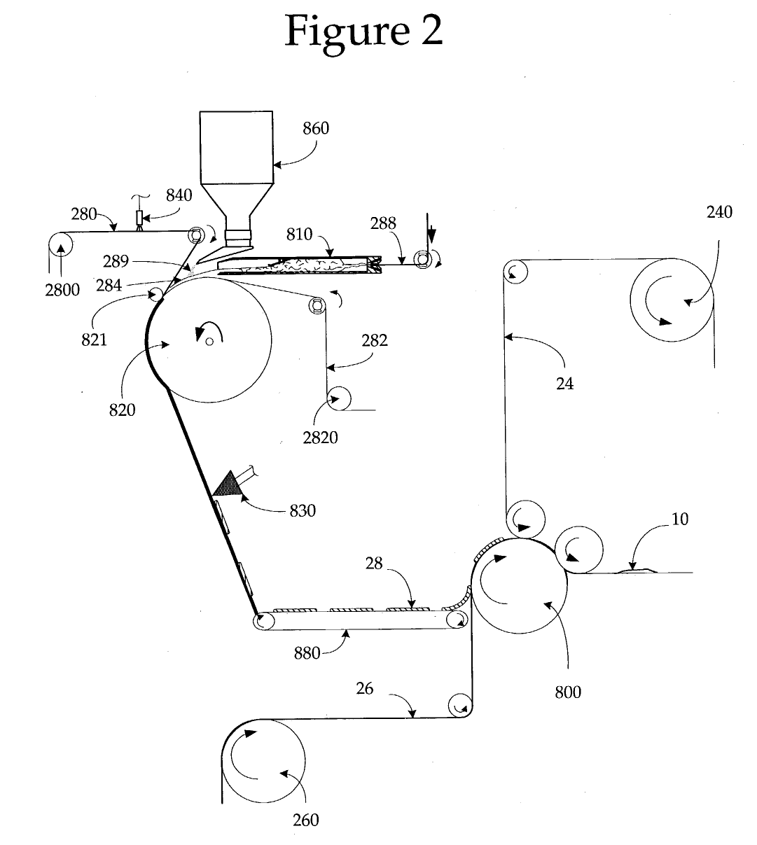

With particular reference to FIG. 2, the absorbent core 28 is formed at core forming station 820 where a central fibrous layer 284, comprised primarily of tow fibers 288 and SAP 289 is disposed between an upper layer 280 and a lower layer 282. The absorbent cores then preferably are individually cut by knife 830 to form absorbent core 28. Additional ingredients may be added to the core at core forming station 820, if desired.

The absorbent core 28 may have a substantially rectangular cross-sectional and plan view shape as shown in FIG. 1, but also may have other shapes, such as a “T” shape or an hourglass shape. The shape of the absorbent core 28 may be selected to provide the greatest absorbency with a reduced amount of material. The absorbent core 28 may be associated with the top sheet 24, back sheet 26, or any other suitable part of the garment 10 by any method known in the art, in order to fix the absorbent core 28 in place. In addition to the respective layers in the absorbent core 28, the overall absorbent core 28 may be enclosed within a tissue wrapping. Skilled artisans are capable of designing and making a suitable absorbent core 28 of the invention, using the guidelines provided herein.

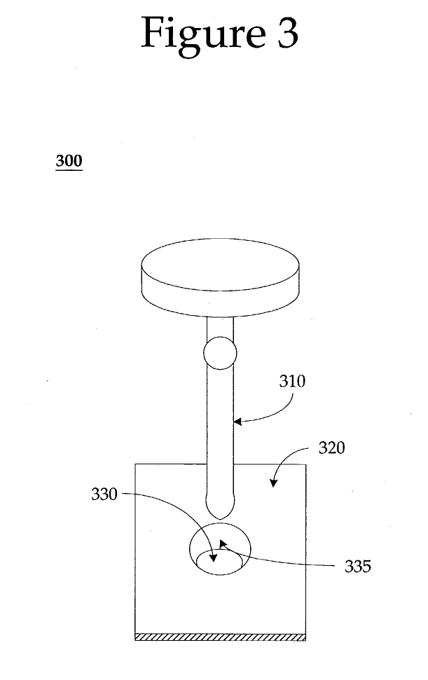

The absorbent core 28 may extend into either or both of the first and second waist regions 12, 14. The absorbent core 28 of one preferred embodiment of the invention preferably includes at least three layers whereby two of the layers are outer layers, (280, 282, FIG. 3) and at least an inner layer that is a central fibrous layer 284 containing a mixture of, inter alia, tow fibers and SAP.

Upper layer 280 and lower layer 282 can be made of any suitable material capable of containing the inner layers (284, 286, etc.) of absorbent core 28. Preferably, upper layer 280 is hydrophilic and fluid pervious, and lower layer 282 is hydrophobic and fluid impervious. More preferably, upper layer 280 and lower layer 282 are comprised of the same tissue-like material. Upper layer 280 and lower layer 282 may be made of the same ply of material folded over the central fibrous layer 284. In this instance, the apparatus of FIG. 2 would be modified to exclude the upper layer 280 material and to include a folding section.

In a preferred embodiment, the central fibrous layer 284 of absorbent core 28 comprises super absorbent polymer distributed within a fibrous structure. Central fibrous layers 284 generally of this type are known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure. It is preferred in the present invention not to fold the absorbent core 28, since folding may cause an increase in stiffness of the absorbent article.

Certain fibrous and particulate additives may be used to maintain high SAP efficiencies when the SAP concentration is in the range of about 50-95%, more preferably about 60-90%, and most preferably about 75-85%. Fibrous additives of central fibrous layer 284 preferably include, but are not limited to, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. Of the foregoing, cellulose acetate is the most preferred fibrous additive for use in central fibrous layer 284. In addition, rayon, Courtauld's LYOCELL, polyacrylonitrile, cotton fibers and cotton linters have similar properties to cellulose acetate and are alternatively preferred. The remaining fibers, surface-modified polyolefin/polyester bicomponent fibers, and surface-modified polyester/polyester bicomponent fibers are also believed to be effective fibrous additives.

The fibrous component of the central fibrous layer 284 most preferably is a crimped tow of cellulose acetate, polypropylene, or polyester, or mixtures thereof. Before making the absorbent core that includes a tow fiber, the tow fiber typically is unwound and opened, and then fed to the core forming station to provide a fibrous mass of material (see, FIG. 2). Skilled artisans are aware of techniques available to open tow fibers and form the opened fibers into a fibrous mass. In addition, the fibrous component of the central fibrous layer 284 may include a low-density roll good made in a separate process. Still further yet, the fibrous component could also include a carded web formed on-line. Optionally, it is advantageous to introduce from about 1-5% of a thermally bondable fiber into the fibrous component of the central fibrous layer 284 for wet strength and core stability in use.

Particulate additives may be added to central fibrous layer 284 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity. hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent composites. Examples of SAP/particulate and SAP/fiber/particulate additives include those described in, for example, U.S. Pat. No. 6,068,620.

Any superabsorbent polymer (SAP) now known or later discovered may be used in central fibrous layer 284, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility. Super absorbent polymers of the surface cross-linked variety are believed to perform best in these absorbent cores of the present invention, especially when used in such high proportions.

Commercially available SAPs include a starch modified superabsorbent polymer available under the trade name HYSORB® from BASF Aktiengesellschaft, Ludwigshafen, Germany. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP, and AQUA KEEP SA60S, manufactured by Sumitomo Seika Chemicals Co., Ltd., Osaka Japan.; ARASORB manufactured by Arakawa Chemical (U.S.A.) Inc.; FAVOR manufactured by Stockhausen Inc.; DIAWET, commercially available from Mitsubishi Chemicals, Japan; FLOSORB, available from SNF Floerger, France, AQUALIC, available from Nippon Shokubai, Osaka, Japan.

In accordance with the present invention, the absorbent core comprises a mixture of tow fibers and SAP. The tow fiber preferably is a continuous crimped filament tow. This fiber structure has high structural integrity, and as such, is distinct from a matrix of discontinuous fibers described as fluff in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff. The low density and thinner absorbent cores contribute to the reduced stiffness of the absorbent articles of the present invention.

The tow fiber can be any continuous or discontinuous thermoplastic filament tow fiber that is capable of being opened and used in combination with SAP in an absorbent core. Preferably, cellulose ester tow is used as the fibrous material in central layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include the ability to absorb moisture, preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994), the disclosure of which is incorporated herein by reference in its entirety.

Continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 25, preferably about 3 to 15. For the same weight product, filaments of lower dpf may provide increased surface area and increased moisture absorption. Total denier may vary within the range of about 5,000 to 60,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and improved performance properties. Gel blocking also may be reduced by using crimped tow in the central layer 284. As therefore may be understood, more crimp is typically better, with an excess of about 20 crimps per inch being usually preferred. Continuous filament, cellulose ester tow having crimped filaments with about 25 to 40 crimps per inch, is commercially available from Celanese Acetate in Charlotte, N.C.

The total basis weights of the absorbent core 28 including fibrous materials, SAP, tissue, additional layer(s), and additives, are anywhere from about 50-1,000 grams per square meter. The most preferred total basis weights of the absorbent core 28 are about 400-700 grams per square meter. Swimmers and feminine care products will have lower basis weights due to less SAP and size, respectively. For example, swimmers typically will have a basis weight in the range of from about 50 to about 350 grams per square meter. Optionally, about 1-10%, preferably about 5%, by weight of thermally bondable synthetic fibers can be added to the absorbent core 28 to impart additional wet strength to the laminate. This will improve the stability of the core during use of the diaper. The preferred synthetic fibers are polyolefin/polyester fibers and polyester/polyester bicomponent fibers.

The foregoing absorbent cores 28 of the preferred embodiments preferably are made using a dry process, whereby the respective components of the composite core 28 are brought together in a dry state, as opposed to one or more components being in a liquid state. Skilled artisans will be capable of making the absorbent cores 28 of the present invention, using the guidelines provided herein.

The method of the invention will be explained in further detail with reference to FIG. 2. The absorbent core 28 preferably is prepared by disposing between an upper layer 280 and a lower layer 282, a central fibrous layer 284 that includes a mixture of tow fibers 288 and SAP 289. While FIG. 2 illustrates upper layer 280 and lower layer 282 being made of two separate materials, the upper and lower layers 280, 282, may be made of the same material, whereby one material is supplied to the core forming station 820, the core is disposed on the layer, and then the layer is folded over onto itself to form an upper and lower layer 280, 282, respectively, comprised of the same material.

The absorbent core 28 may be made by bringing the respective components into contact with one another at a core forming station 820. Upper layer material 280 is provided by upper layer mechanism 2800, and lower layer material 282 is provided by lower layer mechanism 2820. The central fibrous layer is prepared at the core forming station 820 where the SAP particles 289 and opened tow 288 are mixed together. If desired additional layers may be cut and placed within the absorbent core at core forming station 820, using well known cut and place techniques.

Any type of tow fiber 288 can be supplied to the apparatus and, as conventional in the art, the tow fiber 288 typically is opened prior to forming a fibrous matrix. In this regard, the invention preferably includes a tow opener and feeder 810 that is capable of opening any suitable tow material, expanding the tow fiber and feeding the tow fiber to the core forming station 820. Any suitable tow opener and feeder 810 can be used in the method of the invention.

The tow fibers 288 preferably are mixed with superabsorbent polymer (SAP) material 289 to form central absorbent layer 284. The SAP preferably is fed to the core forming station 820 by any SAP feeder 860 capable of feeding the SAP to the core forming station 820. Those skilled in the art are capable of designing a suitable SAP feeder 860 and nozzle configuration (or other dispensing device capable of dispensing and dispersing the SAP in the tow fibers 288) to provide adequate mixing of SAP material 289 and tow fibers 288 to form central fibrous layer 284.

Absorbent core 28 can be formed at core forming station 820, where central fibrous layer 284, comprised of SAP material 289 and tow fibers 288, is disposed between an upper layer 280, and a lower layer 282. Upper and lower layers 280, 282 can be fed to core forming unit 820 using any supplying mechanism known in the art, and preferably are fed through one or more feed rollers 2800, 2820, respectively. Adhesive can be applied to either upper layer 280 or lower layer 282, or to both layers (or to neither layer) by an adhesive applicator 840. Again, any mechanism capable of supplying an adhesive, albeit a spray adhesive, or one that is “rubbed” on, can be used in the invention. Suitable adhesives include any adhesive commonly employed in absorbent garments that is useful in adhering one or more tissue and/or non-woven materials together. It is particularly preferred to use construction adhesives, including HL-1258 by H. B. Fuller Company of St. Paul, Minn.; Findley 2031 and H2587-01 by Ato Findley Inc. of Wauwatosa, Wis.; and NS34-5665 by National Starch Co. of Bridgewater, N.J. Other adhesives that may be used in the invention include 34-578A, available from National Starch Co. of Bridgewater, N.J. Any of these adhesives may be used in all adhesive applications in the absorbent garment, or only in select applications as a construction adhesive for bonding parts of the garment as the top sheet, back sheet, absorbent core, and additional layer(s).

As the SAP material 289 and tow fibers 288 mix together to form central fibrous layer 284, which in turn is disposed between upper layer 280 and lower layer 282 at core forming station 820, some of these SAP particles may become affixed in the adhesive when the absorbent core 28 is passed through the one or more nip rollers 821 at the core forming station 820. The absorbent cores 28 then are cut to length by cutting knife 830. Cutting knife 830 can be any suitable cutting device capable of cutting absorbent core 28 of the invention. For example, cutting knife 830 can be comprised of a set of rollers; one being an anvil, and another having a knife attached at one point on the roller, whereby the diameter of the roller is selected to coordinate with the speed at which absorbent cores 28 are formed. The knife roller and anvil roller then can rotate at the same speed as the line speed to cut the absorbent core 28 at select areas to form uniform length cores 28. Skilled artisans are capable of designing a suitable cutting knife 830 given the specifics of each article forming assembly line.

After forming the absorbent core 28, the cores can be stored for later use, or immediately used as a component part of an absorbent garment. Preferably, the absorbent core 28 is used immediately as a component part of an absorbent garment. In this context, the absorbent cores 28 then would be transported to garment forming station 800 via core conveyor 880 (or any other conveying device) where they will be disposed between a top sheet 24 and a back sheet 26.

Top sheet material 24 may be supplied to forming station 800 by top sheet supply mechanism 240, which can be any supply mechanism capable of supplying top sheet 24 to garment forming station 800. Preferably, top sheet material 24 is supplied via a supply roller 240 and select feed or guide rollers. Back sheet material 26 likewise can be supplied to forming station 800 by back sheet supply mechanism 260, which can be any supply mechanism capable of supplying back sheet 26 to garment forming station 800. Preferably, back sheet material 26 is supplied via a supply roller 260 and select feed or guide rollers. Forming station 800 brings together the respective components of absorbent article 10 by disposing absorbent core 28 between top sheet material 24, and back sheet material 26. The final absorbent article 10 then may be cut and folded to the appropriate size and shape downstream from forming station 800.

The embodiment illustrated in FIG. 2 could and preferably does include a number of additional devices used in preparing absorbent garments. For example, it is conventional to employ leg elastics 30, and standing leg gathers 40. Leg elastics 30 could be supplied to garment forming station 800 by any suitable manner known in the art. It is conventional to apply adhesives to either the leg elastics 30 themselves, or to apply adhesives on back sheet 26 or top sheet 24, and then dispose the leg elastics 30 there between. The leg elastics 30 may be supplied as an elastic element, or as a laminate of elastic elements disposed between two outer layers, preferably outer non-woven materials.

Standing leg gather(s) 40 can be applied as a separate material to top sheet 24, and disposed on the exterior facing side (e.g., body facing side) of the top sheet. The standing leg gathers can be applied either prior to, or downstream from, garment forming station 800 using techniques known in the art.

Fastening elements 34 also can be supplied and attached to garment 10 either prior to or downstream from garment forming station 800. It is preferred that fastening elements are secured to garment 10 after garment forming station 800. It also is preferred that fastening elements 34 include fasteners 36, which can be applied before or after fastening elements 34 are attached to garment 10.

Additional layers also may be placed on garment 10 after forming station 800, or disposed between top sheet 24 and back sheet 26 by supplying the additional layer(s) to garment forming station 800. These additional layer(s) also may be cut-and-placed on top sheet 24 and/or back sheet 26 using known cut-and-place apparatus.

It also is typical in the industry to include a waist elastic system comprising one or more waist elastic materials 32. Waist elastics 32 preferably are supplied upstream of garment forming station 800, and thus disposed between the top sheet 24 and back sheet 26. Waist elastics 32 may, however, be supplied downstream from garment forming station 800, and an outer cover or backing sheet (not shown) disposed outside the back sheet 26, using techniques known in the art. The waist elastic elements 32 may be supplied as a layer of material, or as elastic elements disposed between two outer materials. In a similar fashion, tummy elastics (not shown) may be supplied prior to, or downstream from garment forming station 800.

After the absorbent articles are formed, they typically are packed for ultimate sale to the consumer. Accordingly, FIG. 2 would be modified to include a packing station. Here the individually formed absorbent articles preferably are first folded and then arranged in one or more rows, each containing a plurality of articles. Anywhere from 5-75 articles can be arranged in the one or more rows. The rows of articles 10 then typically are compressed by pressing them tightly, and then packed into an opened carton or bag. The carton or bag is sealed, and preferably prepared for shipping.

Conventional absorbent articles become stiff and relatively uncompressable after packing. After storage, and then opening of the garment, the garment typically will have a stiffness of greater than about 900 gf, and preferably will have a stiffness index of less than about 1.5. In contrast, the present invention provides absorbent articles that have a stiffness of less than 900 gf, even after compression and storage. The absorbent articles of the present invention also preferably have a stiffness index of less than about 1.5.

The present invention therefore relates to absorbent articles such as those described above with reference to FIGS. 1 and 2, whereby the absorbent articles have a stiffness of less than about 900 gf, when measured in accordance with the flexure resistance test using the circular bend procedure, and a stiffness index (stiffness to total capacity ratio) of less than about 1.5 gf/g. More preferably, the absorbent articles of the present invention have a stiffness of less than about 850 gf, even more preferably less than about 800 gf, even more preferably less than about 750, and most preferably less than about 600 gf. The absorbent articles also preferably have a stiffness index of less than about 1.3, even more preferably less than about 1.1, even more preferably less than about 0.9 and most preferably less than about 0.8 gf/g, with the exception of swimmer absorbent articles, in which case the stiffness index is less than about 8.75, more preferably, less than about 8.5, even more preferably, less than about 8.2, and most preferably less than about 8 gf/g.

The present invention also relates to infant diapers, whereby the stiffness is less than about 2,500 gf, with no limitation on the stiffness index. More preferably, the stiffness is less than a bout 2,000 gf, even more preferably less than about 1,400 gf, even more preferably less than about 1,000 gf, and most preferably less than about 750 gf. The total capacity of the infant diaper typically is within the range of from about 200 to about 1,500 grams, more preferably within the range of from about 300 to about 1,200 grams, even more preferably within the range of from about 350 to about 1,000 grams, and most preferably within the range of from about 450 to about 900 grams. The stiffness index of the infant diapers of the present invention preferably is less than about 4.5 gf/g, more preferably less than about 3.5 gf/g, even more preferably less than about 2.0 gf/g, and most preferably less than about 1.0 gf/g.

The present invention also relates pull-on training pants, or “pull-ups,” whereby the stiffness is less than about 900 gf, with no limitation on the stiffness index. More preferably, the stiffness is less than a bout 800 gf, even more preferably less than about 700 gf, even more preferably less than about 600 gf, and most preferably less than about 500 gf. The total capacity of the training pants typically is within the range of from about 200 to about 1,500 grams, more preferably within the range of from about 300 to about 1,200 grams, even more preferably within the range of from about 350 to about 1,000 grams, and most preferably within the range of from about 450 to about 900 grams. The stiffness index of the training pants of the present invention preferably is less than about 1.5 gf/g, more preferably less than about 1.3 gf/g, even more preferably less than about 1.1 gf/g, even more preferably less than about 0.9, and most preferably less than about 0.7 gf/g. The training pants also include adult absorbent articles and diapers, with the exception that the adult products are larger and typically have a higher total capacity. The stiffness and stiffness indices of the adult products, however, fall within the ranges described above with respect to the training pants.

The present invention also relates diapers or training pants intended to be used for swimming, (e.g., “swimmers”), whereby the stiffness is less than about 800 gf, with no limitation on the stiffness index. More preferably, the stiffness is less than a bout 700 gf, even more preferably less than about 625 gf, even more preferably less than about 550 gf, and most preferably less than about 450 gf. Because swimmers typically do not include any SAP, or include very little SAP, the total capacity of the swimmers usually is pretty low, within the range of from about 10 to about 300 grams, more preferably within the range of from about 25 to about 200 grams, even more preferably within the range of from about 40 to about 150 grams, and most preferably within the range of from about 50 to about 100 grams. The stiffness index of the swimmers of the present invention preferably is less than about 9.0 gf/g, more preferably less than about 8.5 gf/g, even more preferably less than about 8.25 gf/g, even more preferably less than about 8.0, and most preferably less than about 7.75 gf/g.

The present invention also relates to methods of making the absorbent articles, each separately having the above-described properties, on average, as well as methods of making a consumer pack containing a plurality of such absorbent articles. The method of making an absorbent article includes providing to a garment forming station a top sheet and a back sheet. The method also include preparing an absorbent core by providing to a core forming station an upper layer, a lower layer, and central absorbent layer comprising a mixture of tow fibers and SAP. The central absorbent layer is formed at the core forming station whereby the tow fibers and SAP are mixed and disposed between the upper and lower layer to form the absorbent core. The method concludes by disposing the absorbent core between the top sheet and the back sheet at the garment forming station.

An alternative embodiment of the invention includes forming the absorbent core at the core forming station by providing a layer of material to the core forming station, forming a central absorbent layer at the core forming station by mixing at least tow fibers and SAP, disposing the central absorbent layer on at least a portion of the layer of material, and then folding the layer of material over the central absorbent layer to provide an absorbent core comprising an upper layer, a lower layer, and a central absorbent layer disposed between the upper and lower layers. In the case of swimmers, SAP typically is not added, or only a small percentage (e.g., less than about 10% by weight based on the weight of the core) to the core forming station.

The invention also includes a method of making a pack of absorbent articles (preferably a consumer pack) comprising making a plurality of absorbent articles in accordance with the method described immediately above. The plurality of absorbent articles then are folded, and assembled adjacent one another to form one or more rows, each containing a plurality of folded absorbent articles. The one or more rows containing a plurality of absorbent articles then are compressed and packaged.

The present invention now will be described with reference to the following non-limiting examples.

### EXAMPLES

The following test methods were used to measure the basis weight, density, stiffness, total capacity, and stiffness index of the absorbent articles tested herein.

Basis Weight and Density

Approximately 5 mean weight articles (diapers, pull-up training pants, swimmers, and feminine napkins) were selected, ensuring that the articles were free of wrinkles, creases, or lumps. The samples were prepared by cutting with scissors the absorbent cores for each of these articles from the remaining portions of the article in such a manner that the top sheet, back sheet, and any other layers that may be present remained in or on the absorbent cores. The sample areas for each of the articles were determined and marked; the sample area being the insult point. The insult point for the diaper and feminine napkin products was the point on the longitudinal centerline that represents the half-way point of the absorbent core from the absorbent article lateral centerline to the edge of the core disposed in the front region of the diaper. The insult point for the training pants and swimmers was at the center of the longitudinal centerline of the core.

A caliper having L and W model 51D20 type SPEC manufactured by AB Lorentzen and Wettre, Stockholm, Sweden, with a pressure foot area of 25 square centimeters and a Mitutoyo precision digital indicator was used to measure the basis weight and density. The caliper was set to zero by pressing the “zero” button, so that the display read 0.00 mm. The pressure foot of the caliper was raised by pressing the button on the side of the caliper to raise the pressure foot high enough to place the sample in the gap. The area under the pressure foot was the marked insult point for each sample. The button then was released to allow the pressure foot to lower onto the sample.

At the first indication that the reading on the thickness gauge was slowing down, the timer was started and set for 30 seconds. At the first sound of the timer alarm, the thickness of the sample was recorded. The sample was released from the caliper, and the balance then was “tared” so that the display read 0.00 g. The sample was placed on the balance and weighed.

The basis weight was calculated in accordance with the equation below:

Basis Weight=Sample Weight(g)/Area of Core(m²)=g/m²

The density was calculated in accordance with the equation 8) below:

Density=Basis Weight(g/m²)/Thickness(mm)/1000=g/cc

The basis weight and density for the absorbent articles then were recorded.

Stiffness

Stiffness was measured using a modified flexure resistance test, utilizing the circular bend procedure, based on the flexure resistance test described in U.S. Pat. No. 5,009,653, (“the '653 patent”) the disclosure of which is incorporated by reference herein in its entirety. The flexure resistance test described in the '653 patent was used to measure the stiffness of sanitary napkins, which typically are much thinner and smaller than diapers, training pants, or swimmers. The test method therefore was modified by enlarging the orifice on the plate platform and increasing the size of the plunger on the Instron apparatus.

The flexure resistance test described herein is a simultaneous, multidirectional deformation of an absorbent product in which one face of the specimen becomes concave and the other face becomes convex. This process is known as a Circular Bend Procedure. The Circular Bend Procedure, and it provides a force value related to flexure-resistance, simultaneously averaging stiffness in all directions.

The apparatus useful in carrying out the stiffness test of the present invention is illustrated in FIG. 3. As shown in FIG. 3, the Instron apparatus 300, preferably an Instron Model No. 4411 or equivalent, commercially available from Instron Engineering Corporation, Canton, Mass., includes a plunger 310, a platen 320 including an orifice 330, whereby the edges 335 of the orifice 330 are chamfered at a 45° angle.

The Instron computer control software, Series IX, was set to “Compressive Test Method #08, and the 5.0 Kg load cell was used. The platen 320 was about 102×102×6.35 mm, and the orifice 330 had a diameter of about 37.5 mm. The plunger 310 had an overall length of about 84 mm, a diameter of about 7.5 mm, and a ball nose having a radius of 0.14 mm. The plunger was mounted concentric with the orifice having an equal clearance on all sides. The bottom of the plunger was set well above the top of the orifice plate, at or about 12.7 mm from the top of platen 320.

The samples prepared and measured above were subjected to the stiffness testing. The examples below tested diapers, training pants and swimmers, where the diapers were Stage 4 diapers and the training pants were large. As mentioned above, each sample was cut so that only the core portion remained, making sure not to cut into the core and expose the contents. The core portion included the top sheet, the absorbent core, and the back sheet, and any other additional layers that may have remained.

The Instron was set to “Compressive Test Method #8”. Each sample was placed squarely on top of the platen orifice 330 with the “body side” facing up (i.e., the top sheet 24 is facing up). Each sample was positioned with its predetermined test point (marked above as the insult point) directly under the plunger. The Instron then was activated so that the plunger would slowly contact the sample and begin bending it. The Instron Series IX program recorded and reported the average of peak force values. The settings for the Instron Series IX Compressive Method are set forth in the Table below.

Settings for Series IX Compressive Method #8

| | |
|---|---|
| Machine Control | Relaxation |
| Speed | 500 mm/min |
| Test Direction | Down |
| Action | Stop & Return |
| Limit Type | Displacement |
| Limit Value | 18.95 mm |
| Calculation | # 1.2 Max Load |

The average peak force was recorded as the stiffness in grams-force (gf).

Total Capacity

The following equipment and reagants were used.

1.0% sodium chloride solution

Permanent marker

2 plastic tubs (approximately 8″ deep×18″ wide×24″ long) or equivalent

Plastic basket to fit inside tub, approximately 1-2″ tall

2 stainless steel weights, approximately 1150 g each

Plastic tray

Timer

Drain stand

Metal binder clips

Digital balance, with 100 g load and 0.1 g resolution

Platform balance, with 5000 g load and 2 g resolution

Plexiglas tray, dimensions larger than an open diaper

Sample Preparation:

The same five samples for each absorbent article used to measure the stiffness also were used to measure the total capacity.

Test Procedure:

Each article was weighed on the digital balance, and the weight recorded to the nearest 0.1 g. One tub was filled to about ¾ full with 1.0% saline. The first sample was positioned so that it lay flat with the top sheet 24 side facing up. This sample was slowly placed in the tub of saline solution, such that the sample was completely de-aerated. This procedure was repeated with the remaining samples.

The plastic basket then was placed on top of the samples and one or two weights were placed on the basket to keep the articles submerged. The timer was started and set for 60 minutes (one hour). After one hour, each sample then was hung from the draining stand using two binder clips, and the timer again was started and set this time for two minutes. After two minutes had elapsed, the samples were removed and placed on a dry plastic tray.

The dry plastic tray was carried to the balance, and the plexiglas tray was placed on the platform balance. The balance was “tared” so the weight measured 0.0, and then the first sample was weighed and the weight recorded to the nearest gram. The remaining samples were weighed, making sure each time to zero the balance before each new weight to account for any liquid that may have been left on the plexiglas from the previous article.

The total capacity of each sample was calculated for each sample, and the average taken for the five samples. The total capacity was determined in accordance with the equation below:

Capacity(g)=Wet diaper weight(g)−Dry diaper weight(g)

Stiffness Index

The stiffness index was determined by dividing the stiffness by the total capacity, and is reported in gf/g.

### Examples and Comparative Examples

A number of commercially available popular brand diapers, pull-up training pants and swimmers were purchased for comparison purposes. These articles were compared with diapers, training pants and swimmers prepared in accordance with the present invention. The absorbent core for the diapers and training pants were essentially the same, whereas the absorbent core for the swimmers contained no superabsorbent polymer.

The absorbent articles of the present invention were prepared in a conventional manner, except that the absorbent core was modified in the following manner. The absorbent core for the inventive diaper 1 was prepared by disposing between two conventional tissue sheets approximately 12% by weight 2.7 denier cellulose acetate tow, and about 88% by weight SAP commercially available from BASF (BASF 7200). Conventional glue was used to adhere the tissue sheets together. The absorbent core for the inventive diaper 2 was prepared in the same manner as inventive diaper 1, except the tow was a 7.3 denier cellulose acetate tow.

The inventive training pant was prepared in a conventional manner except the absorbent core was modified by disposing between two conventional tissue sheets approximately 15% by weight 7.3 denier cellulose acetate tow, and approximately 85% by weight BASF 3900 SAP. The inventive swimmers were prepared in the same manner as the inventive training pants, except no SAP was used in the core.

Samples of the comparative articles and the inventive articles then were tested in accordance with the basis weight, density, stiffness, and total capacity tests described above. The results are shown in the Table below.

| | Basis | | Total | Stiff- | Stiffness |
|---|---|---|---|---|---|
| | Weight | Density | Capacity | ness | Index |
| Product | (gsm) | (g/cc) | (g) | (gf) | (gf/g) |
| Diaper Comp. 1* | 1071.7 | 0.16 | 633.0 | 4251.4 | 6.716 |
| Diaper Comp. 2 | 766.6 | 0.09 | 658.1 | 3075.0 | 4.673 |
| Diaper Comp. 3 | 752.4 | 0.10 | 570.8 | 2799.0 | 4.904 |
| Inventive Diaper 1 | 833.0 | 0.19 | 830.7 | 573.8 | 0.691 |
| Inventive Diaper 2 | 827.9 | 0.20 | 679.4 | 521.6 | 0.768 |
| Training pants | 618.2 | 0.10 | 619.2 | 942.0 | 1.521 |
| Comp. 1 | | | | | |
| Training pants | 613.3 | 0.13 | 684.4 | 1056.1 | 1.543 |
| Comp. 2 | | | | | |
| Inventive Training | 634.4 | 0.16 | 661.0 | 405.4 | 0.613 |
| Pant | | | | | |
| Swimmers Comp. 1 | 478.9 | 0.11 | 90.5 | 812.6 | 8.979 |
| Inventive Swimmers | 321.3 | 0.08 | 52.4 | 403.2 | 7.695 |

*Comp. denotes comparison.

As seen from the above table, the inventive diapers, training pants, and swimmers had improved stiffness, when compared to commercially available comparative absorbent articles. The inventive absorbent articles also had similar or better total capacity that provides an improved stiffness index.

Conventional Ultra Thin feminine napkins also were tested in accordance with the procedures described above. The feminine napkins either contained a non-woven outer sheet, or a 3-dimensional apertured sheet.

Five samples of each of the articles were prepared as described above, and the insult points marked. The results of the tests are shown in the Table below.

| | | | | | Stiff- |
|---|---|---|---|---|---|
| | | | Total | Stiff- | ness |
| | Basis Weight | Density | Capacity | ness | Index |
| Product | (gsm) | (g/cc) | (g) | (gf) | (gf/g) |
| Fem Comp. 1 NW | 351.5 | 0.16 | 61.3 | 653.6 | 10.66 |
| Fem Comp. 2-3D | 345.9 | 0.13 | 64.4 | 730.6 | 11.35 |
| Fem Comp. 3 NW | 357.3 | 0.15 | 58.4 | 675.1 | 11.56 |
| Fem Comp. 4-3D | 308.9 | 0.13 | 27.3 | 719.4 | 26.35 |

One would expect feminine napkin products to have a lower stiffness when compared to training pants and diapers, as disclosed in the '653 patent. The stiffness index, however, is higher than the stiffness index of any of the absorbent articles of the present invention.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent article comprising a top sheet, a back sheet, and an absorbent core at least partially disposed between the top sheet and the back sheet, whereby the absorbent article has a stiffness of less than about 900 gf, and a stiffness index of less than about 1.5 gf/g.

2. The article of claim 1, wherein the absorbent core is comprised of an upper layer, a lower layer, and a central fibrous layer disposed between the upper layer and lower layer.

3. The article of claim 2, central fibrous layer comprises from about 30% to about 95% by weight super absorbent polymer (SAP).

4. The article of claim 2, wherein the central fibrous layer comprises tow fibers selected from the group consisting of cellulose ester fibers, polypropylene fibers, and mixtures thereof.

5. The article of claim 4, wherein the tow fibers are cellulose acetate tow fibers.

6. The article of claim 2, wherein the upper and lower layer are comprised of the same material, and the material is selected from the group consisting of tissue, airlaid fluff pulp, synthetic non-woven material, and mixtures or combinations thereof.

7. The article of claim 6, wherein the upper layer is fluid pervious, and the lower layer is fluid impervious.

8. The article of claim 2, wherein the central fibrous layer further comprises up to 10% by weight fluff wood pulp fibers.

9. The article of claim 2, wherein the central fibrous layer further comprises particulate additives.

10. The article of claim 1, wherein the stiffness is less than about 850 gf.

11. The article of claim 1, wherein the stiffness is less than about 750 gf.

12. The article of claim 1, wherein the stiffness is less than about 600 gf.

13. The article of claim 1, wherein the stiffness index is less than about 1.3 gf/g.

14. The article of claim 1, wherein the stiffness index is less than about 1.1 gf/g.

15. The article of claim 1, wherein the stiffness index is less than about 0.8 gf/g.

16. A plurality of absorbent articles that have been folded and arranged in a consumer pack, each absorbent article comprising a top sheet, a back sheet, and an absorbent core at least partially disposed between the top sheet and the back sheet, whereby the plurality of absorbent articles have an average stiffness of less than about 900 gf, and an average stiffness index of less than about 1.5 gf/g.

17. An absorbent article comprising a top sheet, a back sheet, and an absorbent core at least partially disposed between the top sheet and the back sheet, whereby the absorbent article has a stiffness index (ratio of stiffness to total capacity) of less than about 1.5 gf/g.

18. The article of claim 17, wherein the absorbent core is comprised of an upper layer, a lower layer, and a central fibrous layer disposed between the upper layer and lower layer.

19. The article of claim 18, central fibrous layer comprises from about 30% to about 95% by weight super absorbent polymer (SAP).

20. The article of claim 18, wherein the central fibrous layer comprises tow fibers selected from the group consisting of cellulose ester fibers, polypropylene fibers, and mixtures thereof.

21. The article of claim 20, wherein the tow fibers are cellulose acetate tow fibers.

22. The article of claim 18, wherein the upper and lower layer are comprised of the same material, and the material is selected from the group consisting of tissue, airlaid fluff pulp, synthetic non-woven material, and mixtures or combinations thereof.

23. The article of claim 22, wherein the upper layer is fluid pervious, and the lower layer is fluid impervious.

24. The article of claim 18, wherein the central fibrous layer further comprises up to 10% by weight fluff wood pulp fibers.

25. The article of claim 18, wherein the central fibrous layer further comprises particulate additives.

26. The article of claim 17, wherein the stiffness index is less than about 1.3 gf/g.

27. The article of claim 17, wherein the stiffness index is less than about 1.1 gf/g.

28. The article of claim 17, wherein the stiffness index is less than about 0.8 gf/g.

29. A plurality of absorbent articles that have been folded and arranged in a consumer pack, each absorbent article comprising a top sheet, a back sheet, and an absorbent core at least partially disposed between the top sheet and the back sheet, whereby the plurality of absorbent articles have an average stiffness index of less than about 1.5 gf/g.

30. A diaper comprising a top sheet, a back sheet, and an absorbent core at least partially disposed between the top sheet and the back sheet, whereby the absorbent article has a stiffness of less than about 2,500 gf.

31. The diaper of claim 30, wherein the absorbent core is comprised of an upper layer, a lower layer, and a central fibrous layer disposed between the upper layer and lower layer.

32. The diaper of claim 31, central fibrous layer comprises from about 30% to about 95% by weight super absorbent polymer (SAP).

33. The diaper of claim 31, wherein the central fibrous layer comprises tow fibers selected from the group consisting of cellulose ester fibers, polypropylene fibers, and mixtures thereof.

34. The diaper of claim 33, wherein the tow fibers are cellulose acetate tow fibers.

35. The diaper of claim 31, wherein the upper and lower layer are comprised of the same material, and the material is selected from the group consisting of tissue, airlaid fluff pulp, synthetic non-woven material, and mixtures or combinations thereof.

36. The diaper of claim 35, wherein the upper layer is fluid pervious, and the lower layer is fluid impervious.

37. The diaper of claim 31, wherein the central fibrous layer further comprises up to 10% by weight fluff wood pulp fibers.

38. The diaper of claim 31, wherein the central fibrous layer further comprises particulate additives.

39. The diaper of claim 30, wherein the stiffness is less than about 2,000 gf.

40. The diaper of claim 30, wherein the stiffness is less than about 1,000 gf.

41. The diaper of claim 30, wherein the stiffness is less than about 750 gf.

42. The diaper of claim 30, wherein the diaper has a stiffness index of less than about 4.5 gf/g.

43. The diaper of claim 42, wherein the stiffness index is less than about 3.5 gf/g.

44. The diaper of claim 43, wherein the stiffness index is less than about 1.0 gf/g.

45. A plurality of diapers that have been folded and arranged in a consumer pack, each diaper comprising a top sheet, a back sheet, and an absorbent core at least partially disposed between the top sheet and the back sheet, whereby the plurality of diapers have an average stiffness of less than about 2,500 gf.

46. A training pant comprising a top sheet, a back sheet, and an absorbent core at least partially disposed between the top sheet and the back sheet, whereby the absorbent article has a stiffness of less than about 900 gf.

47. The training pant of claim 46, wherein the absorbent core is comprised of an upper layer, a lower layer, and a central fibrous layer disposed between the upper layer and lower layer.

48. The training pant of claim 47, central fibrous layer comprises from about 30% to about 95% by weight super absorbent polymer (SAP).

49. The training pant of claim 47, wherein the central fibrous layer comprises tow fibers selected from the group consisting of cellulose ester fibers, polypropylene fibers, and mixtures thereof.

50. The training pant of claim 49, wherein the tow fibers are cellulose acetate tow fibers.

51. The training pant of claim 47, wherein the upper and lower layer are comprised of the same material, and the material is selected from the group consisting of tissue, airlaid fluff pulp, synthetic non-woven material, and mixtures or combinations thereof.

52. The training pant of claim 51, wherein the upper layer is fluid pervious, and the lower layer is fluid impervious.

53. The training pant of claim 47, wherein the central fibrous layer further comprises up to 10% by weight fluff wood pulp fibers.

54. The training pant of claim 47, wherein the central fibrous layer further comprises particulate additives.

55. The training pant of claim 46, wherein the stiffness is less than about 700 gf.

56. The training pant of claim 46, wherein the stiffness is less than about 600 gf.

57. The training pant of claim 46, wherein the stiffness is less than about 500 gf.

58. The training pant of claim 46, wherein the training pant has a stiffness index is less than about 1.5 gf/g.

59. The training pant of claim 58, wherein the stiffness index is less than about 1.3 gf/g.

60. The training pant of claim 59, wherein the stiffness index is less than about 0.7 gf/g.

61. A plurality of training pant s that have been folded and arranged in a consumer pack, each training pant comprising a top sheet, a back sheet, and an absorbent core at least partially disposed between the top sheet and the back sheet, whereby the plurality of training pants have an average stiffness of less than about 900 gf.

62. A swimming pant comprising a top sheet, a back sheet, and an absorbent core at least partially disposed between the top sheet and the back sheet, whereby the absorbent article has a stiffness of less than about 800 gf.

63. The swimming pant of claim 62, wherein the absorbent core is comprised of an upper layer, a lower layer, and a central fibrous layer disposed between the upper layer and lower layer.

64. The swimming pant of claim 63, central fibrous layer comprises from about 0% to about 5% by weight super absorbent polymer (SAP).

65. The swimming pant of claim 63, wherein the central fibrous layer comprises tow fibers selected from the group consisting of cellulose ester fibers, polypropylene fibers, and mixtures thereof.

66. The swimming pant of claim 65, wherein the tow fibers are cellulose acetate tow fibers.

67. The swimming pant of claim 63, wherein the upper and lower layer are comprised of the same material, and the material is selected from the group consisting of tissue, airlaid fluff pulp, synthetic non-woven material, and mixtures or combinations thereof.

68. The swimming pant of claim 67, wherein the upper layer is fluid pervious, and the lower layer is fluid impervious.

69. The swimming pant of claim 63, wherein the central fibrous layer further comprises up to 10% by weight fluff wood pulp fibers.

70. The swimming pant of claim 63, wherein the central fibrous layer further comprises particulate additives.

71. The swimming pant of claim 62, wherein the stiffness is less than about 700 gf.

72. The swimming pant of claim 62, wherein the stiffness is less than about 550 gf.

73. The swimming pant of claim 62, wherein the stiffness is less than about 450 gf.

74. The swimming pant of claim 62, wherein the swimming pant has a stiffness index is less than about 9.0 gf/g.

75. The swimming pant of claim 74, wherein the stiffness index is less than about 8.25 gf/g.

76. The swimming pant of claim 75, wherein the stiffness index is less than about 7.75 gf/g.

77. A plurality of swimming pant s that have been folded and arranged in a consumer pack, each swimming pant comprising a top sheet, a back sheet, and an absorbent core at least partially disposed between the top sheet and the back sheet, whereby the plurality of swimming pants have an average stiffness of less than about 800 gf.

78. A method of making an absorbent article comprising:
providing to a garment forming station a top sheet and a back sheet;
preparing an absorbent core by providing to a core forming station an upper layer, a lower layer, and central absorbent layer comprising a mixture of tow fibers and SAP;
forming the central absorbent layer at the core forming station whereby the tow fibers and SAP are mixed and disposed between the upper and lower layer to form the absorbent core; and
disposing the absorbent core between the top sheet and the back sheet, wherein the absorbent article has a stiffness of less than about 900 gf, and optionally a stiffness index of less than about 1.5 gf/g.

79. The method of claim 78, whereby the absorbent article is prepared such that the top sheet and the back sheet form a first waist region, a second waist region longitudinally opposite the first waist region, and a crotch region between the waist regions.

80. The method of claim 79, further comprising
e) attaching at least one fastening element to lateral edges of the first waist region; and
f) preparing at least one target device and attaching the at least one target device to the article in the second waist region, where the at least one fastening element and the at least one target device are capable of attaching to one another, the at least one target device being located so that the first waist region and second waist region of the article may be joined to one another to secure the article on a wearer.

81. The method of claim 80, wherein the at least one fastening element comprises a hook portion of a hook and loop fastener and the at least one target device comprises the loop portion of a hook and loop fastener.

82. The method of claim 80, wherein the at least one fastening element is an adhesive tape and the at least one target device comprises a tape receiving surface.

83. The method of claim 80, wherein the at least one fastening element is comprised of a pair of laterally extending tabs disposed on the lateral edges of the first waist region, whereby the laterally extending tabs each include at least one fastening element.

84. The method of claim 78, wherein the central fibrous layer comprises from about 30% to about 95% by weight super absorbent polymer (SAP).

85. The method of claim 78, wherein the central fibrous layer comprises tow fibers selected from the group consisting of cellulose ester fibers, polypropylene fibers, and mixtures thereof.

86. The method of claim 85, wherein the tow fibers are cellulose acetate tow fibers.

87. The method of claim 78, wherein the upper and lower layer are comprised of the same material, and the material is selected from the group consisting of tissue, airlaid fluff pulp, synthetic non-woven material, and mixtures or combinations thereof.

88. The method of claim 87, wherein the upper layer is fluid pervious, and the lower layer is fluid impervious.

89. The method of claim 78, wherein the central fibrous layer further comprises up to 10% by weight fluff wood pulp fibers.

90. The method of claim 78, wherein the stiffness is less than about 850 gf.

91. The method of claim 78, wherein the stiffness is less than about 750 gf.

92. The method of claim 78, wherein the stiffness is less than about 600 gf.

93. The method of claim 78, wherein the stiffness index is less than about 1.3 gf/g.

94. The method of claim 78, wherein the stiffness index is less than about 1.1 gf/g.

95. The method of claim 78, wherein the stiffness index is less than about 0.8 gf/g.

96. A method of making a pack of absorbent articles comprising:
preparing a plurality of absorbent articles in accordance with claim 78;
folding the plurality of absorbent articles;
assembling the plurality of absorbent articles adjacent one another to form one or more rows, each containing a plurality of folded absorbent articles; and
compressing and packaging the one or more rows containing a plurality of absorbent articles.
